(19) United States
(12) Patent Application Publication
(10) Pub. No.: US 2007/0010756 A1
(43) Pub. Date: Jan. 11, 2007

(54) Patient monitoring during drug administration

(76) Inventor: Hanna E. Viertio-Oja, Espoo (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/176,945

(22) Filed: Jul. 7, 2005

(51) Int. Cl. A61B 5/04

(52) U.S. Cl. 600/544

(57) ABSTRACT

The invention relates to a method and apparatus for monitoring a patient during drug administration. In the monitoring process, at least one state index indicative of the clinical state of the patient is determined based on physiological signal data obtained from the patient. In order to eliminate inconsistencies caused by varying drug combinations in the at least one state index, drug administration data is maintained, which identifies the drug(s) administered to the patient, and the determination of the at least one state index is controlled in dependence on the drug administration data, thereby to produce values for the at least one state index, which remain substantially consistent regardless of the particular combination of the drugs administered to the patient.

### FIELD OF THE INVENTION

The present invention relates generally to patient monitoring during drug administration. The invention finds a typical application in patient monitors monitoring the extent of the hypnotic state of a patient during anesthesia.

### BACKGROUND OF THE INVENTION

Neuromonitoring is a subfield of clinical patient monitoring focused on measuring various aspects of brain function and on changes therein caused by drugs commonly used to induce and maintain anesthesia in an operation room or sedation in patients under critical or intensive care.

Electroencephalography (EEG) is a well-established method for assessing brain activity by recording and analyzing the weak biopotential signals generated in the cortex of the brain with electrodes attached on the skin of the skull surface. The EEG has been in wide use for decades in basic research of the neural systems of the brain as well as in the clinical diagnosis of various neurophysiological diseases and disorders.

Electromyography (EMG) is a method for recording electrical biopotentials of muscles. In an EMG measurement, the electrodes are attached on top of a muscle onto the surface of the skin. When an EEG signal is recorded from the forehead of the patient, the recorded signal indicates both the activity of the facial muscles (fEMG) and the brain (EEG).

One of the special applications of the EEG, which has received attention recently, is the use of a processed EEG signal for objective quantification of the amount of brain activity for the purpose of determining the level of consciousness of a patient. In its simplest form, the utilization of an EEG signal allows automatic detection of the alertness of an individual, i.e. if he or she is awake or asleep. This has become an issue of increased interest, both scientifically and commercially, in the context of measuring the depth of unconsciousness induced by anesthesia during surgery.

Another important component of balanced anesthesia is analgesia which means prevention of pain reactions of a patient by administration of pain medication. Adequate analgesia reduces surgical stress and there is firm evidence that it decreases postoperative morbidity. Awareness during surgery with insufficient analgesia may lead to a post-traumatic stress disorder. Low quality pre- and intra-operative analgesia makes it difficult to select an optimal pain management strategy later on. More specifically, it may cause exposure to unwanted side effects during the recovery from the surgery. If the anesthesia is too light and involves insufficient hypnosis, it may cause traumatic experiences both for the patient and for the anesthesia personnel. From an economical point of view, if the anesthesia is too deep, it may cause increased perioperative costs through extra use of drugs and time, and extend the time required for post-operative care.

Virtually every patient being cared for in an Intensive Care Unit, for example, receives some form of sedation. However, the control of the depth of the sedation administered to a patient is still problematic, and therefore oversedation and undersedation are both common occurrences in ICUs. At present, monitoring the level of sedation is mainly handled by using subjective observations from the patient. Various sedation assessment scales have been developed for subjectively assessing the level of sedation, the Ramsay Score being one of the most widely used tools for this purpose. Inappropriate sedation can lead to adverse clinical outcomes and reduce efficiencies in critical care settings. Oversedation may cause various complications, such as cardiovascular instability, and it may also increase the length of stay in the hospital and prolong the usage time of expensive facilities, such as the intensive care unit. Undersedation, in turn, may result in patient anxiety and agitation, which can further interfere with care and result in harm to the patient and the nursing staff.

In addition to the EEG signal data, EMG signal data obtained from facial muscles (fEMG) of the forehead is used for monitoring purposes during anesthesia and intensive care. Recovering facial muscle activity is often the first indicator of the patient approaching consciousness. When this muscle activity is sensed by electrodes placed appropriately, it provides an early indication that the patient is emerging from anesthesia. Similarly, these electrodes can sense pain reactions when the anesthesia is not adequate due to inadequate analgesia. So, EMG signals give an early warning of arousal and may also be indicative of inadequate analgesia.

The depth of hypnosis is not directly measurable. Therefore, drug delivery systems have to derive the level of hypnosis from a surrogate signal or from indirectly measured parameters. The most common and popular surrogate signal for this purpose is the EEG (electroencephalogram), from which several parameters may be determined. The basic reason for the insufficiency of a single parameter is the variety of drugs and the complexity of the drug effects on the EEG signal in human brains. However, during the past few years, some commercial validated devices for measuring the level of consciousness and/or awareness in clinical set-up during anesthesia or sedation have become available. Two of these devices, which are based on a processed EEG signal and examine the signal as a whole with its multiple features, are marketed by GE Healthcare Finland Oy, Kuortaneenkatu 2, 00510 Helsinki, Finland (Entropy Index) and by Aspect Medical Systems, Inc., 141 Needham Street, Newton, Mass. 02464, U.S.A. (Bispectral Index, BIS™).

In the S/5 Entropy Module of GE Healthcare Finland Oy, two entropic indicators termed State Entropy (SE) and Response Entropy (RE) are computed. State Entropy, which primarily reflects the cortical state of the patient, is computed over a frequency range from 0.8 Hz to 32 Hz, which corresponds to the EEG-dominant part of the spectrum. The Response Entropy, in turn, is computed over a frequency range from 0.8 Hz to 47 Hz, which also contains EMG frequencies. The difference between the State Entropy and the Response Entropy is then indicative of the EMG activation. A combined indication provided by the State Entropy and the said entropy difference is then used to assess the level of hypnosis or sedation. The S/5 Entropy Module is based on the mechanisms described in U.S. Pat. No. 6,801,803, which is incorporated herein by reference in its entirety. The entropy calculation algorithm of the S/5 Entropy Module has also been described in Viertiö-Oja H, Maja V, SärkeläM, Talja P, Tenkanen N, Tolvanen-Laakso H, Paloheimo M, Vakkuri A, Yli-Hankala A, Meriläinen P: Description of the Entropy algorithm as applied in the Datex-Ohmeda S/5 Entropy Module, Acta Anaesthesiologica Scandinavica 2004; Volume 48: Issue 2: 154-161, 2004.

The BIS involves the calculation of three subparameters, BetaRatio, SyncFastSlow, and Burst Suppression, and the resulting index is a combination of the three subparameters. Some of the techniques for analyzing EEG signals in an effort to determine the depth of anesthesia as well as the principles of the BIS algorithm are described in Ira J. Rampil, A Primer for EEG Signal Processing in Anesthesia, Anesthesiology, Vol. 89(4) October 1998, pp. 980-1002.

The above-mentioned commercially validated devices thus rest on spectral entropy and BIS, respectively, which are commonly perceived as measures of the hypnotic component of anesthesia. It is therefore to be expected that patients lose their consciousness at the same entropy or BIS value independently of the anesthetic drug that has been used.

However, a recent study shows that this is not exactly the case but the value corresponding to the loss of consciousness (LOC) depends on the particular drug combination for both entropy and BIS values. More particularly, the said study indicates that if an analgesic is used in addition to a hypnotic drug, the entropy or BIS value corresponding to the LOC is higher than the value if only the hypnotic drug is used, see Struys M., et al., British Journal of Anesthesia, 93(5): 645-54 (2004). The observed drug-dependence may result from the different action mechanisms of the drugs, giving rise to different EEG patterns.

The present invention seeks to alleviate or eliminate the above-mentioned drawback.

### SUMMARY OF THE INVENTION

The present invention seeks to decrease or eliminate the inconsistencies that a varying drug combination may cause in a measure indicative of the clinical state of the patient. The clinical state here refers to the physiological status of the patient or a particular organ of the patient, where the said status is indicative of a need or effect of a treatment or intervention and where the term physiological relates to physiology, the science dealing with the functions of living matter and beings. As discussed below, the clinical state typically refers to the depth of hypnosis. The invention further seeks to provide a mechanism for producing consistent measures of the clinical state for all drugs and drug combinations that may be used when the clinical state of the patient is monitored.

In the present invention, drug administration data is maintained which describes desired features of the current drug administration process, and a monitoring process that determines at least one measure of the state of the patient is controlled in dependence on the said data so that the at least one measure remains substantially consistent regardless of the combination of drugs administered to the patient. This means that consistent values are obtained for the at least one measure even if the drug combinations used vary, or that the deviations from the consistent values remain so small that it is irrelevant in terms of the treatment of the patient.

Thus one aspect of the invention is providing a method for monitoring a patient. The method includes the steps of administering at least one drug to a patient, maintaining drug administration data that identifies the at least one drug administered in the administering step, and obtaining physiological signal data from the patient. The method further includes the steps of determining, based on the physiological signal data, at least one state index indicative of a clinical state of the patient, and controlling the determining step in dependence on the drug administration data, thereby to produce values for the at least one state index, which remain substantially consistent regardless of the at least one drug administered to the patient.

Another aspect of the invention is that of providing an apparatus for monitoring a patient. The apparatus comprises means for receiving drug administration data that identifies at least one drug administered to a patient, monitoring means for determining, based on physiological signal data obtained from the patient, at least one state index indicative of a clinical state of the patient, and control means for controlling the monitoring means in dependence on the drug administration data, thereby to produce values for the at least one state index, which remain substantially consistent regardless of the at least one drug administered to the patient.

The present invention enables consistent and comparable measures of the clinical state to be produced during an entire monitoring process, such as a surgery, regardless of the varying drug combinations used during the process.

In one embodiment, drug-dependent inconsistencies are eliminated by applying a correction transform to the calculated measures of the clinical state. In another embodiment, the process determining the said measures is controlled so that the calculated measures are substantially free of the drug-dependent inconsistencies.

A further aspect of the invention is that of providing a computer program product by means of which known patient monitoring devices may be upgraded and thus their accuracy improved. The program product includes a first program code portion configured to receive drug administration data which identifies at least one drug administered to a patient and a second program code portion configured to control a monitoring process adapted to calculate at least one state index indicative of a clinical state of the patient, wherein the second program code portion is configured to control the monitoring process in dependence on the drug administration data thereby to eliminate drug-dependent inconsistencies in the at least one state index.

Other features and advantages of the invention will become apparent by reference to the following detailed description and accompanying drawings.

### BRIEF DESCRIPTION OF THE DRAWINGS

In the following, the invention and its preferred embodiments are described more closely with reference to the examples shown in FIG. 1 to 5 in the appended drawings, wherein:

FIG. 1 is a schematic diagram showing one embodiment of a patient monitoring apparatus or system of the invention;

FIG. 2 is a schematic diagram showing a modification of the embodiment shown in FIG. 1;

FIG. 3 illustrates one embodiment of the control carried out based on drug administration data;

FIG. 4 illustrates another embodiment of the control carried out based on drug administration data; and

FIG. 5 illustrates one embodiment of the system according to the invention.

### DETAILED DESCRIPTION OF THE INVENTION

The above-mentioned study (Struys M., et al.) thus shows that the value corresponding to loss of consciousness depends on the drug combination for both spectral entropy and BIS. In the study, the BIS and the SE and RE values were defined during propofol infusion with and without remifentanil. Propofol is a hypnotic agent, while remifentanil is an opiod. Both are commonly used as anesthetic agents.

The study indicates that for propofol only, LOC takes place at a lower entropy/BIS value than for the combination of propofol and remifentanil. The table below shows the values of State Entropy, Response Entropy, and BIS at which 50 percent of the patients had lost consciousness (95 percent confidence interval). The first column shows the results for a group of patients anesthetized with propofol only, while the second and third columns show the corresponding results for patient groups in which constant remifentanil effect-site concentrations of 2 and 4 ng/ml, respectively, were maintained with step-by-step increase of propofol concentration.

| | remi 0 ng/ml | remi 2 ng/ml | remi 4 ng/ml |
|---|---|---|---|
| SE | 64 (62-66) | 68 (67-69) | 70 (68-72) |
| RE | 70 (68-72) | 74 (73-75) | 81 (80-83) |
| BIS | 61 (59-62) | 68 (67-70) | 71 (70-73) |

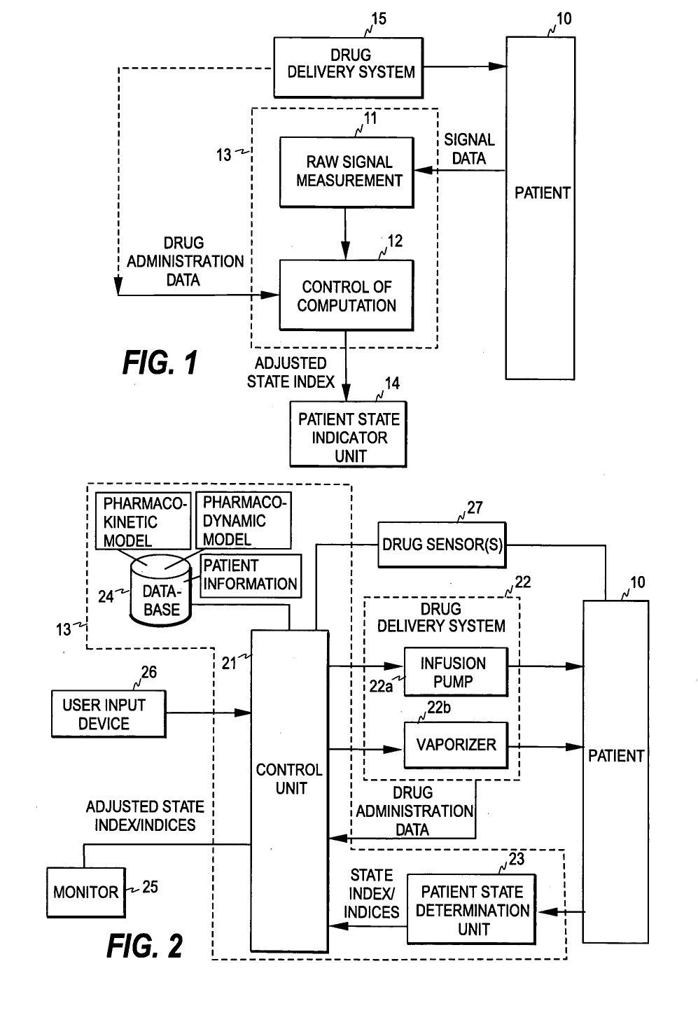

Although the discrepancy between the patient groups in the above results is considerable, the confidence intervals are relatively tight: inside each patient group both entropy and BIS estimate LOC accurately. It is therefore possible, by combining the information on the current drug combination with the EEG measurement, to obtain an index for the depth of hypnosis, which remains consistent for any drug combination and optimally accurate for any drug. This is discussed first with reference to FIG. 1, which shows the adjustment of the invention removing drug-dependent inconsistencies in the index values.

As noted above, the monitoring device of the invention produces, based on the physiological signal data obtained from the patient, at least one measure of the relevant clinical state of a patient. Below, the said measure is termed the state index. Although the state index is typically indicative of the depth of hypnosis or sedation and thus also of the overall state of the patient, it may also be indicative of the physiological state of a particular organ of the patient. The clinical state may thus also refer to the physiological state of a particular organ. The process that produces the at least one state index may at a logical level be divided into two parts. In the first part 11 the at least one state index may be defined in a conventional manner, i.e. as if the varying drug combinations did not produce any inconsistency in the at least one state index. The second part 12 in turn controls the calculation of the at least one state index based on drug administration data indicating current values for predetermined properties of the drug administration process, such as the drug(s) and the respective concentrations administered. Together the first and second parts form a measurement unit 13 that outputs at least one state index which remains consistent regardless of the drug combination supplied from a drug delivery system 15 to the patient. The said at least one state index is shown to the user through a display or another appropriate indicator unit 14. There are two basic implementation alternatives for the measurement unit 13. In the first implementation, the first and second parts may be separate and successive parts, as shown in the figure, whereby the first part determines the at least one state index and supplies it/them to the second part, which adjusts the said index/indices based on the drug administration data. In this implementation, the measurement unit performs a correction transform that converts the original state index values to output values that remain consistent regardless of a change in the drugs administered. In the second implementation, the second part may be embedded in the first part, whereby the second part controls the internal operation of the first part to enable the first part to output one or more state indices which remain consistent regardless of the drug combination. In the second implementation, the measurement unit maps the current drug administration data to certain control parameter values used for the determination of the state indices, whereby the state index values output from the measurement unit are substantially free of drug-dependent inconsistencies.

In a typical monitoring process, such as a surgery, the second part operates dynamically in response to the drug administration data, since the drug administration characteristics may vary substantially during the process. However, in another application the adjustment/control carried out by the second part may be less dynamic.

The drug administration data may be received from a drug delivery system 15, which administers drugs to the patient at the same time as the measurement unit determines the said at least one state index. The content of the drug administration data is at its minimum when only one drug is administered to the patient, in which case the said data may include only the identifier of the drug in question.

As is common in the art, the digitized signal data obtained from the patient is processed as sets of sequential signal samples representing finite time blocks or time windows, commonly termed “epochs”. The length of the epoch thus determines the minimum time interval required to update the value of each state index. However, the time intervals at which the current drug administration data is taken into account is not necessarily as short. While the state index is typically updated at an interval of a few seconds, the drug administration data may be taken into account 2 to 5 times in a minute, for example. Furthermore, the correction transform or the control parameter values do not necessarily have to be updated at regular intervals, but the update may be carried out only when the drug administration data indicates a sufficient change in the drug administration process.

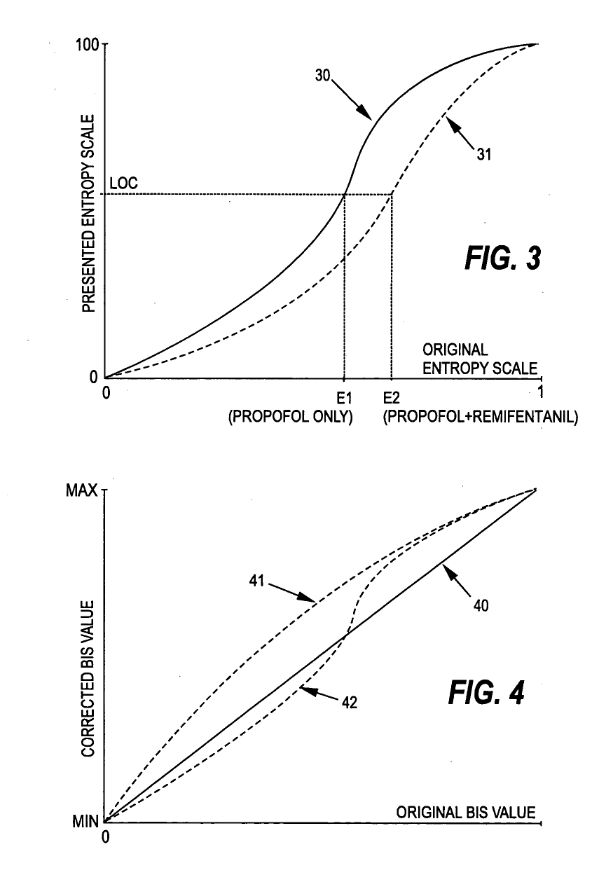

FIG. 2 shows one modification of the patient monitoring system of FIG. 1. In this case, the measurement unit 13 of FIG. 1 comprises a patient state determination unit 23 and a control unit 21 provided with a database 24. The patient state determination unit is typically a hypnosis index determination unit outputting at least one entropy value or a BIS value. These values are supplied to the control unit, which continuously receives drug administration data from a drug delivery system 22. The drug delivery system is typically an anesthesia delivery unit, which may comprise an intravenous infusion pump 22a for intravenously administered drugs and/or a vaporizer 22b for inhaled drugs. Based on the drug administration data, the control unit adjusts the entropy value(s) or the BIS value obtained from the patient state determination unit. To this end, the control unit knows or is able to define the correction transform needed to convert the entropy/BIS values received to corrected output entropy/BIS values supplied to a monitor. The correction transforms needed may be implemented, for example, by parameterized, drug-dependent scaling functions, as is discussed below in connection with FIGS. 3 and 4. The drug administration data received by the control unit thus unambiguously determines the correction transform needed to convert the input entropy/BIS values to corrected output entropy/BIS values. In one embodiment, the current drug(s) and the respective concentration(s) indicated by the drug administration data directly determine the correction transform. However, the control unit may also utilize a pharmacokinetic model and possibly also a pharmacodynamic model for determining the correction transform.

A pharmacokinetic model is a mathematical model describing how a certain drug is distributed in the course of time from the site of delivery to different parts of the body and to a particular organ in which the drug is supposed to have its effect. Thus, pharmacokinetics describes what a body does to the drug. Because of the complexity of the physiology of the body, the models are typically based on theoretical body compartments, such as plasma, fat, or brain. Pharmacokinetic models typically also employ anthropometric data, such as patient height, weight, age, gender, etc. Pharmacodynamics, in turn, describes what a certain drug does to the body, i.e. a pharmacodynamic model describes the relationship between a drug concentration and its effect.

The control unit may therefore be provided with a database 24 storing the pharmacokinetic and/or the pharmacodynamic models for the drugs available. The database may also store the patient information utilized by the models. Based on the drug(s) used, currently administered drug concentration(s) C_{adm}(t), and various anthropometric data, such as the weight and gender of the patient, the control unit may first determine, using the pharmacokinetic model, the drug concentration(s) in the brain C_{brain}(t). The obtained brain concentration may then be used to define the correction transform needed. Depending on the measurement, the control unit may also use a pharmacodynamic model.

As shown in FIG. 2, the system may also comprise additional measurement units 27 for measuring drug concentrations or for enhancing patient safety. A typical measurement unit that is used in connection with inhaled drugs is a sensor for measuring the end tidal drug concentration exhaled by the patient. The feedback signal provided by the sensor is useful in the sense that it enables a simpler pharmacokinetic model to be used in the control unit when brain concentration is estimated. This is because the concentration of a drug in the end tidal breathing gases of the patient corresponds to the concentration in the lungs, which is very close to the concentration in the blood circulation of the patient. Knowing the concentration in the blood circulation in turn allows for the use of a simpler pharmacokinetic model to obtain the concentration in the brain.

As noted above, the drug administration data obtained from the drug delivery system includes at least the identifier(s) of the drug(s) administered at each time. The drug administration data typically also includes the information indicative of the amount of drug(s) administered, such as fresh gas concentration if a volatile gas is administered, or infusion rate (ml/min) if an infusible drug is used. If a volatile gas is administered, the control unit may also receive the inspired and/or expired gas concentration(s) measured. Using the incoming information and applying the pharmacokinetic model(s) the control unit may then determine the effect-site concentrations. By applying the pharmacodynamic models the control unit may further estimate the effects of the drug administration.

The drug delivery system may also be an intelligent unit which may perform some of the above operations of the control unit. For example, the drug delivery unit may have access to the pharmacokinetic and/or pharmacodynamic model(s), whereby it may determine the effect-site concentrations and/or the effects of the drug administration. In this case the drug administration data thus includes processed information, which reduces the amount of computations needed in the control unit. The intelligence of the system may thus be distributed between different units of the system.

FIG. 3 illustrates one embodiment of the correction transform carried out by the control unit in the embodiment of FIG. 2. In this case, the patient state determination unit is a hypnosis index determination unit outputting at least one spectral entropy value, such as the RE and SE values. In order to enhance the resolution of the patient monitor in the range of clinical anesthesia and emergence, current entropy-based patient monitors employ a nonlinear transformation to transform the calculated entropy values on the original entropy scale [0 . . . 1] to an another integer scale, such as [0 . . . 100]. In this way, the resolution of the patient monitor is the best possible in the most interesting range of adequate hypnosis and emergence, which lies above entropy values of about 0.5. The nonlinear transformation may be implemented by a spline function, which is denoted by reference numeral 30 in FIG. 3. As can been from the figure, the slope of the function is highest in the middle region. In one embodiment of the invention, this modification of the known patient monitors is employed to convert the original entropy values to entropy values free of drug-dependent inconsistencies by replacing the spline scaling function 30 by a drug-dependent scaling function whenever needed. If only a hypnotic is administered, the entropy value corresponding to the loss of consciousness (LOC) may be, for example, E1, as is shown in the figure. However, if an analgesic is also administered, the entropy value corresponding to the LOC point may be, for example, E2 (where E2>E1). In this case, the basic scaling function is replaced by a drug-dependent scaling function 31, which corrects the entropy value so that the entropy value presented to the user (y-axis) remains substantially the same.

Since the drug administration data now indicates that an analgesic is involved, the control unit defines, based on the analgesic and its current concentration, the correct transform 31, which keeps the entropy value corresponding to the LOC point substantially the same as without the analgesic.

As shown in FIG. 4, the same principle may be used to transform the BIS values if the patient state determination unit 23 is based on a BIS algorithm. If no analgesics are used, the transform 40 corresponds to a straight line that does not change the original BIS value calculated in the patient state determination unit. However, if an analgesic is administered, the control unit determines the correction transform 41, 42 that corresponds to the said analgesic and its current concentration.

The exact forms of the drug-dependent transforms may be determined experimentally, and the transforms may be implemented as parameterized function transforms in which the parameters are derived based on the drug administration data. One possible parameter that may be used to define the transform needed in each case is the ratio of the concentration of the analgesic to the concentration of the hypnotic. If this ratio is zero, the control unit uses the basic scaling function and if the ratio is greater than zero, the control unit defines the correction transform that corresponds to the current value of the ratio. As noted above, the concentrations used may be delivery-site or effect-site concentrations.

It is possible to perform clinical experiments in which the dependence of the relation of the state index and the clinical state of the patient is determined in a systematic way. For example, the value of the state index may be measured for the following six clinical states (corresponding to the Ramsay Sedation Scale) using varying combinations of drugs:
1. Patient is anxious and agitated or restless, or both.
2. Patient is co-operative, oriented, and tranquil.
3. Patient responds to commands only.
4. Patient exhibits brisk response to light glabellar tap or loud auditory stimulus.
5. Patient exhibits a sluggish response to light glabellar tap or loud auditory stimulus.
6. Patient exhibits no response.

For the measurement to work consistently, the range of values of the state index in any of these different clinical states should be consistent for the drug combination being used. The correction transform or the transform of the control parameter values is then obtained by implying this constraint (only the discrete points corresponding to the discrete clinical states can be fixed and extrapolation is used between the fixed points.)

A set of transform curves may also be defined, each optimized for a particular drug. When more than one drug is administered simultaneously, a linear combination of the corresponding curves may be defined by retrieving the coefficients corresponding to the particular drug combination. The dependence of the coefficients of the linear combination on the drugs may be experimentally determined in advance.

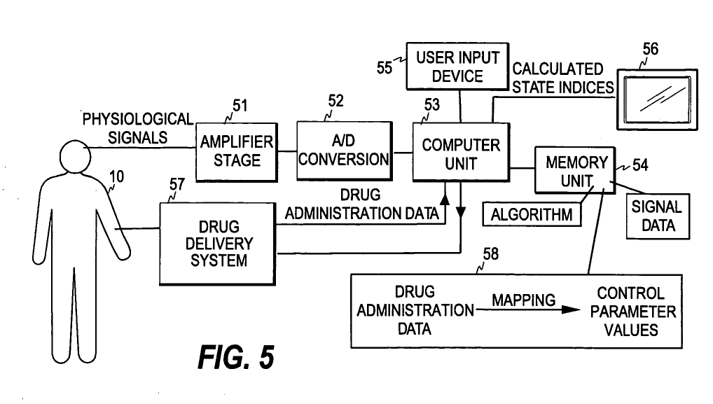

FIG. 5 illustrates one embodiment of the system or apparatus according to the invention. The physiological signal(s) obtained from one or more sensors attached to a patient 10 are supplied to an amplifier stage 51, which amplifies the signal(s) before they are sampled and converted into digitized format in an A/D converter 52. The digitized signals are supplied to a computer unit 53 which may comprise one or more processors. As noted above, the signal data measured from the patient is typically EEG signal data, which is measured through electrodes applied to the forehead of the patient. The electrodes normally also receive EMG signal data from the patient.

The computer unit is provided with a memory or database 54 holding the digitized signal data obtained from the sensor(s). The memory or database may also store the algorithm used for determining at least one state index indicative of the clinical state of the patient, such as the entropy or BIS algorithm for determining the depth of hypnosis.

In this embodiment, no drug-dependent adjustment is needed for the calculated state indices, but the state indices output by the computer unit can be directly supplied to a monitor 56 since they are already consistent. To output such state indices, the control unit continuously receives drug administration data from a drug delivery system 57 and maps the said data to values of predetermined control parameters, which are used in the algorithm to calculate the at least one state index. The mapping may be carried by a look-up table 58, for example, as is shown in the figure. The algorithm thus employs drug-dependent parameters for the calculation of the said at least state index and the control unit controls the values of the said parameters, and possibly also the operation of the algorithm, in dependence on the drug administration data. This is discussed below assuming that computer unit determines spectral entropies indicative of the depth of hypnosis during anesthesia.

When spectral entropies are calculated, the respective frequency range may be a drug-dependent parameter. As noted above, in the S/5 Entropy Module of the Applicant State Entropy is computed over a frequency range from 0.8 Hz to 32 Hz and the Response Entropy over a frequency range from 0.8 Hz to 47 Hz.

When a pure hypnotic is administered, the computer unit may compute the SE and RE values normally. Various drugs may, however, affect the EEG spectrum in different ways. Ketamine, for example, induces high frequency activity. Therefore, in case ketamine is administered, a single entropy value may be calculated over a frequency range wider than the frequency ranges used in case of pure hypnotics. The upper limit of the said single frequency range may be increased to a certain first limit value, which is substantially above the conventional EEG frequency range. In this case, the single entropy value is indicative of the cortical state of the patient, because the frequency band used covers practically the whole EEG frequency range.

In one embodiment of the invention, the control of the parameters may be carried out by applying weights to various spectral components and the values of the weights may be varied depending on the drug combination.

Although one computer unit or processor may perform the above steps, the processing of the data may also be distributed among different units/processors (servers) within a network, such as a hospital LAN (local area network). The apparatus of the invention may thus also be implemented as a distributed system.

The computer unit may further act as a controlling entity controlling the administration of the drugs from the delivery system 57 to the patient. The computer unit may also supply the state index to another computer unit or microprocessor (not shown), which then acts as the controlling entity controlling the drug delivery system. The said controlling entity may be provided with the control data needed for the administration, such as the pharmacodynamic and pharmacokinetic properties of the drugs to be administered. The drug delivery system may comprise separate delivery units for one or more drugs to be administered, such as delivery unit for an analgesic drug and/or a delivery unit for a hypnotic drug.

The computer unit may also act as decision-support tool for the physician, such as an anesthesiologist, who may control the operation of the drug delivery system through an appropriate user input device 55, such as a keyboard or a bar code reader. Various parameters possibly needed in the calculation of the state indices may also be supplied through the input device, if the computer unit has no access to such data.

A conventional patient monitor may also be upgraded to enable the monitor to determine a state index, which remains consistent regardless of the drug combinations administered to the patient. Such an upgrade may be implemented by delivering to the patient monitor a software module that enables the device to control the calculation of the state index in the above-described manner. The software module may be delivered, for example, on a data carrier, such as a CD or a memory card. The software module is provided with a first input interface configured to receive drug administration data from a drug delivery system. In the above-mentioned first implementation, the software module is further provided with a second input interface for receiving the state indices determined by the patient monitor. The software module then performs the transforms illustrated in FIGS. 3 and 4 and outputs state indices that remain substantially consistent for any drug combination. In the above-mentioned second implementation, the software module comprises the said first input interface and an output interface through which the module controls the internal operation of the patient monitor, such as the frequency ranges over which spectral entropies are calculated and/or the weights of the spectral components.

Instead of entropy, the evaluation of the level of hypnosis may be based on another parameter that characterizes the amount of disorder or irregularity in the input EEG signal data. Other possible quantifications that may be used include fractal spectrum analysis, Lempel-Ziv complexity, or bispectral or multispectral analyses, and the method of the invention may be used to adjust the state index defined by any existing patient monitor. As a more detailed discussion of the various mathematical techniques available can be found in the above-referred U.S. Pat. No. 6,801,803, these methods are not discussed in detail in this context.

Although the above-described control and adjustment mechanisms are mainly intended for the determination of the depth of hypnosis, they may be applied in connection with the determination of any state index indicative of the clinical state of the patient. For example, a patient monitor may output a state index indicative of the patient's level of nociception (where nociception refers to conscious or unconscious perception of physiological pain), and the correction mechanism discussed above may be used to make the index of nociception substantially free of inconsistencies caused by varying drug combinations. Furthermore, the mechanism may also be used in connection with future drugs that may affect the measured physiological signal differently than the current drugs. For example, new anesthetics may be introduced, which give rise to an EEG spectrum that deviates from the EEG spectrum caused by the anesthetics commonly used today. The mechanism of the invention may then be utilized to yield a consistent measure of the depth of hypnosis regardless of the type of the anesthetic used.

Although the invention was described above with reference to the examples shown in the appended drawings, it is obvious that the invention is not limited to these, but may be modified by those skilled in the art without departing from the scope and spirit of the invention.

1. A method for monitoring a patient, the method comprising the steps of:
administering at least one drug to a patient;
maintaining drug administration data that identifies the at least one drug administered in the administering step;
obtaining physiological signal data from the patient;
based on the physiological signal data, determining at least one state index indicative of a clinical state of the patient; and
controlling the determining step in dependence on the drug administration data, thereby to produce values for the at least one state index, which remain substantially consistent regardless of the at least one drug administered to the patient.

2. A method according to claim 1, wherein the controlling step includes controlling the determining step dynamically in dependence on the drug administration data, whereby the values remain substantially consistent regardless of the at least one drug administered to the patient at each time.

3. A method according to claim 2, wherein
the obtaining step includes obtaining EEG signal data from the patient; and
the determining step includes determining the at least one state index, in which the at least one state index is indicative of the hypnotic level existing in the patient.

4. A method according to claim 3, wherein determining step includes determining the irregularity of the EEG signal data.

5. A method according to claim 3, wherein determining step includes determining the spectral entropy of the EEG signal data.

6. A method according to claim 3, wherein the determining step includes determining a bispectral value based on a bispectral analysis.

7. A method according to claim 2, wherein the controlling step includes a step of correcting the at least one state index in dependence on the drug administration data.

8. A method according to claim 1, wherein the controlling step includes a step of changing at least one parameter value used for determining the at least one state index in the determining step, wherein the changing step is performed in dependence on the drug administration data.

9. A method according to claim 7, wherein the correcting step includes the sub-steps of defining a correction transform based on the drug administration data and applying the correction transform to the at least one state index.

10. A method according to claim 9, wherein
the administering step includes administering an opioid to the patient;
the maintaining step includes maintaining drug administration data,
in which the drug administration data further indicates the amount of the opioid administered in the administering step; and
the defining sub-step includes defining the correction transform based on the opioid and the amount indicated by the drug administration data.

11. A method according to claim 8, wherein the changing step includes changing at least one frequency range used in the determining step.

12. A method according to claim 7, wherein the determining step includes determining the at least one state index, in which the at least one state index is indicative of the patient's level of nociception.

13. An apparatus for monitoring a patient, the apparatus comprising:
means for receiving drug administration data that identifies at least one drug administered to a patient;
monitoring means for determining, based on physiological signal data obtained from the patient, at least one state index indicative of a clinical state of the patient; and
control means for controlling the monitoring means in dependence on the drug administration data, thereby to produce values for the at least one state index, which remain substantially consistent regardless of the at least one drug administered to the patient.

14. An apparatus according to claim 13, wherein the control means are configured to control the monitoring means dynamically in dependence on the drug administration data, whereby the values remain substantially consistent regardless of the at least one drug administered to the patient at each time.

15. An apparatus according to claim 14, wherein the monitoring means are configured to receive EEG signal data from the patient and to determine, based on the EEG signal data, at least one state index indicative of the hypnotic level existing in the patient.

16. An apparatus according to claim 15, wherein the monitoring means are configured to determine the irregularity of the EEG signal data.

17. An apparatus according to claim 16, wherein the monitoring means are configured to determine the spectral entropy of the EEG signal data.

18. An apparatus according to claim 15, wherein the monitoring means are configured to determine a bispectral value based on a bispectral analysis.

19. An apparatus according to claim 14, wherein control means are configured to correct the at least one state index in dependence on the drug administration data.

20. An apparatus according to claim 13, wherein control means are configured to change at least one parameter in dependence on the drug administration data, and wherein the at least one parameter is used by the monitoring means for determining the at least state index.

21. An apparatus according to claim 19, wherein the control means are further configured to define a correction transform based on the drug administration data and apply the correction transform to the at least one state index.

22. An apparatus according to claim 21, wherein
the drug administration data indicates the amount of an opioid administered to the patient; and
the control means are further configured to define the correction transform based on the opioid and the amount.

23. An apparatus according to claim 20, wherein control means are configured to change at least one frequency range in dependence on the drug administration data, the at least one frequency range being used by the monitoring means for determining the at least state index.

24. An apparatus according to claim 13, further comprising drug delivery means for delivering the at least one drug to the patient, the drug delivery means being configured to produce the drug administration data.

25. An apparatus for monitoring a patient, the apparatus comprising:
a first controller configured to receive drug administration data that identifies at least one drug administered to a patient;
a computing unit configured to determine, based on physiological signal data obtained from the patient, at least one state index indicative of a clinical state of the patient; and
a third controller configured to control the computing unit in dependence on the drug administration data, thereby to produce values for the at least one state index, which remain substantially consistent regardless of the at least one drug administered to the patient.

26. A computer program product for an apparatus monitoring a patient, the computer product comprising:
a first program code portion configured to receive drug administration data which identifies at least one drug administered to a patient; and
a second program code portion configured to control a monitoring process adapted to calculate at least one state index indicative of a clinical state of the patient, wherein the second program code portion is configured to control the monitoring process in dependence on the drug administration data thereby to eliminate drug-dependent inconsistencies in the at least one state index.
